(19) United States
(12) Patent Application Publication
(10) Pub. No.: US 2008/0147171 A1
(43) Pub. Date: Jun. 19, 2008

(54) Synthetic Prosthesis for Aortic Root Replacement

(75) Inventors: Timothy Rawden Ashton, West Kilbride (GB); Roshan Maini, Bridge of Weir (GB); Paul Burns, Glasgow (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: VASCUTEK LIMITED, Inchinnan (GB)

(21) Appl. No.: 11/663,569

(22) PCT Filed: Oct. 7, 2005
(86) PCT No.: PCT/GB05/03883

§ 371 Date: Mar. 23, 2007

(30) Foreign Application Priority Data
Oct. 7, 2004 (GB) 0422231.1

(51) Int. Cl. A61F 2/06; B65D 81/24

(52) U.S. Cl. 623/1.24; 623/1.39; 623/1.4; 600/36; 206/524.4

(57) ABSTRACT

There is described a synthetic aortic conduit formed from two tubular porous layers having a non-bioresorbable sealant, such as SEPs, interposed therebetween. The synthetic aortic conduit can be attached to an aortic valve, such as a xenograft valve, to form an aortic root replacement prosthesis. The synthetic aortic conduit has the advantage that it can be stored in the preservative solutions required for tissue valves without degradation.

The present invention relates to a synthetic aortic conduit, an aortic root replacement prosthesis comprising the synthetic aortic conduit, and the use of the aortic root replacement prosthesis in a method of treating heart disease. Particularly, but not exclusively, the present invention relates to a synthetic aortic conduit comprising a non-bioresorbable sealant.

The preferred method of treating disease affecting the aorta and/or the aortic valve is to replace the entire aortic root (Bentall et al., Thorax, 1968, 23:338-9) and this requires implantation of a replacement aortic valve and aortic conduit. A variety of devices have been used for aortic root replacement (ARR), including xenograft (tissue) valves, mechanical valves, xenograft conduits and synthetic conduits.

Commonly a graft comprising a mechanical valve and a synthetic conduit is used. The valve is typically carbon and bi-leaflet, and the conduit is typically formed from woven polyester. In order to minimise blood loss the woven polyester conduit is preferably sealed with a bioresorbable sealant. This may be gelatin (Luciani et al., Ann Thorac Surg, 1999, 68:2258-62), or collagen (Girardi et al., Ann Thorac Surg, 1997, 64:1032-5). Several problems are associated with the implantation of mechanical aortic valves. In particular, a patient having an implanted mechanical aortic valve requires systemic anticoagulation treatment to prevent clotting and embolisation induced by the non-natural surface and flow characteristics of the mechanical valve.

The use of valves formed from tissue (autografts from cadavers or xenografts) is also known in the art. The xenografts are typically either animal valves used intact (usually porcine) or are made from pericardium (usually bovine) fabricated into a valve. In both cases the material, which is largely collagen, has to be stabilised against degradation. This is achieved by cross-linking, usually with glutaraldehyde.

The haemocompatability of the collagen surface and the more natural haemodynamics of tissue valves eliminates the need for systemic anticoagulation treatment on implantation. The major limitation associated with xenograft valves is their restricted durability. The collagen becomes the subject of calcification and biodegrades after a relatively short time period. However, improvements in tissue processing and anti-calcification treatments have extended the useful life of tissue valves to the point where their use has overtaken mechanical valves, and the proportion of tissue based grafts implanted continues to grow.

Unlike mechanical valves, tissue valves cannot be stored in a dry environment. To maintain the collagen structure and cross-linking, the tissue valves are stored in a preservative solution. This is usually a dilute solution of glutaraldehyde or formaldehyde.

As described above, synthetic aortic conduits are known and are usually the conduits of choice for use during valve replacement. The synthetic aortic conduits are commonly formed from woven polyester, and are attached to the aortic valve with a bioresorbable sealant. Known synthetic aortic conduits of this type are incompatible with the preservative solutions used to store tissue valves, as known bioresorbable sealants react with the preservative solutions needed for the tissue valves. The cross-linking of the bioresorbable sealant is typically increased on exposure to the preservative solution, affecting the bioresorption rate of the sealant. Modifying the preservative solution to make it compatible with the conduit sealant is problematic, as the long term effects on the tissue valve have to be taken into account. Any solution which does not affect the sealant, is unlikely to preserve the valve adequately.

To avoid the problems associated with an altered bioresorption rate, synthetic conduits are usually only sealed to the tissue valves at the point of use, i.e. the time of implantation. This is inconvenient and introduces unnecessary delays in the surgical procedures, thereby increasing risk to the patient.

We have now found an aortic conduit which is compatible with the storage conditions for a tissue valve. Consequently the present invention provides an aortic conduit, as well as a combined prosthesis comprising a tissue valve already attached to the conduit.

Previously, it has always been considered that the use of a non-bioresorbable sealant for synthetic aortic conduits would prevent tissue ingrowth into the conduit, and this would complicate healing. However, we have now found that a non-bioresorbable sealant can be used in an aortic conduit.

According to a first aspect of the present invention there is provided a synthetic aortic conduit comprising:
i) a first inner tubular layer formed of a porous material;
ii) a second outer tubular layer formed of a porous material; and
a non-bioresorbable sealant layer interposed between said first and second tubular layers.

The non-bioresorbable sealant is selected to be compatible with a preservative solution used to store tissue valves, and preferably the synthetic aortic conduit remains unaffected by prolonged exposure to the preservative solution. Consequently, a prosthesis comprising a tissue valve and a synthetic aortic conduit attached thereto may be stored in a standard preservative solution until required. The ability to store the aortic conduit under the same conditions as the tissue valve enables the conduit and valve to be pre-attached together to form an aortic root replacement prosthesis prior to storage, thereby avoiding the time delays and inconvenience of attaching the synthetic aortic conduit to the tissue valve at the point of use. The need for anti-coagulants associated with the use of mechanical aortic valves is also avoided.

Preferably the synthetic aortic conduit does not react with, and is non-bioresorbable in, preservative solutions comprising aldehydes such as solutions of glutaraldehyde and/or formaldehyde.

The synthetic aortic conduit of the present invention has the advantage that the porous open structure of the two tubular layers retains the ability for tissue ingrowth from both sides of the graft.

The first and second tubular layers may be formed from the same material or from different materials. The materials forming the first and second tubular layers may have the same or different porosities.

The porous material used in forming either one or both of the tubular layers of the conduit is conveniently polyester, suitably knitted or woven polyester. Other suitable porous materials include ePTFE (expanded polytetrafluoroethylene).

The porous materials of the first and second tubular layers are not required to have a permeability to resist leakage. In the conduit, water or blood tightness is provided by the sealant layer which is typically formed from a non-porous impermeable layer of elastomer.

In one embodiment the first tubular layer which forms the inner luminal surface of the synthetic aortic conduit is in contact with the patient's blood.

In one embodiment the first inner tubular layer is formed of a material having a porosity sufficient to permit tissue ingrowth and ensure good attachment of the pseudointima. The water permeability of the inner tubular layer will be high, generally 4,000 to 10,000 ml/min/cm² at 120 mmHg pressure. For most applications, a permeability of 8,000 to 10,000 ml/min/cm² at 120 mmHg will be suitable.

In one embodiment, the second outer tubular layer is formed from a porous material having sufficient porosity to permit tissue ingrowth, although this is less critical than for the first inner layer. In one embodiment the water permeability of the second outer tubular layer is 1,000 to 3,000 ml/min/cm² at 120 mmHg. For most applications a water permeability of 1,800 to 2,000 ml/min/cm² at 120 mmHg is suitable.

Optionally the first inner tubular layer has a water permeability of 4,000 to 10,000 ml/min/cm² at 120 mmHg pressure and the second outer tubular layer has a water permeability of 1,000 to 3,000 (preferably 1,800 to 2,000) ml/min/cm² at 120 mmHg pressure.

In one embodiment the second outer tubular layer provides dimensional stability to the graft and will resist radial expansion.

In one embodiment the material of the second outer tubular layer is a woven material and the material of the first inner tubular layer is a knitted material.

In a separate embodiment the first and second tubular layers are both formed from knitted material.

Optionally both the first and second tubular layers are formed from polyester. Thus the synthetic aortic conduit may comprise a knitted polyester first inner tubular layer and a woven polyester second outer tubular layer. Alternatively the layers could each be of knitted polyester.

Suitably the non-bioresorbable sealant is biocompatible, flexible and durable. Preferably the non-bioresorbable sealant exhibits good binding to the first and second tubular layers. The non-bioresorbable sealant is generally a polymer, typically an elastomeric polymer such as silicone, polyurethane and polyester thermoplastic elastomers. Styrene-olefin block copolymers are of particular interest, especially a styrene-ethylene-propylene-styrene copolymer, optionally plasticised with squalane (SEPS). The SEPS material has excellent chemical resistance and is unaffected by the storage solutions used for tissue heart valves.

In one embodiment the synthetic aortic conduit is in the form of a “sandwich” construction having three or more layers. Thus the synthetic aortic conduit may comprise an outer woven tubular layer, an inner knitted layer and a non-bioresorbable sealant layer formed from SEPS interposed there between.

Suitably the synthetic aortic conduit is shaped to mimic the sinuses of Valsalva (see U.S. Pat. No. 6,352,554; EP 955019). This may improve the haemodynamics and leaflet motion of the aortic view.

Optionally, the graft is crimped.

In a second aspect of the present invention there is provided a method of forming a synthetic aortic conduit, said method comprising:
a) interposing a non-bioresorbable sealant layer between a first tubular layer and a second tubular layer to form a composite structure;
b) heating the composite structure of step a) at 50 to 150° C. for 15 to 60 minutes to form the synthetic conduit.

In one embodiment step b) involves heating the composite structure of step a) at 110° C. for 30 minutes.

In one embodiment the composite structure of step a) is formed inside out with the outer tubular surface of the composite structure of step a) forming the inner luminal surface of the synthetic aortic conduit formed. In this embodiment the method will additionally include the step of reversing the composite structure (ie. turning it inside out) so the outer tubular surface of the composite structure becomes the inner layer of the reversed composite structure and vice versa. The reversal step suitably takes place after step b).

Optionally the aortic conduit is then crimped. Crimping may conveniently be achieved by spirally winding a beading onto the outer surface of the conduit followed by heating (eg. 50 to 150° C. for 30 to 90 minutes).

Suitably the method may comprise the steps of:
a) interposing a non-bioresorbable sealant layer between a first tubular layer and a second tubular layer to form a composite structure;
b) heating the composite structure of step a) at 50 to 100° C. for 15 to 60 minutes;
c) reversing the composite structure so the inner tubular layer becomes the outer tubular layer and vice versa;
d) wrapping a spiral winding around the exterior of the composite structure at a first pitch;
e) heating the spirally wound composite structure of step d) at 40 to 80° C. for 30 to 60 minutes;
f) heating the wrapped composite structure of step e) at 100 to 150° C. for 10 to 30 minutes; and
g) removing the spiral winding from the composite structure.

Suitably step b) comprises heating the composite structure of step a) at 110° C. for 30 minutes.

Suitably step e) comprises heating the spirally wound composite structure of step d) at 60° C. for 40 minutes.

Suitably step f) comprises heating the spirally wound composite structure of step e) at 110° C. for 20 minutes.

According to a further aspect of the present invention there is provided the synthetic aortic conduit as described above for use in therapy, particularly for use in the treatment of heart conditions, heart disease and/or defects, suitably those which affect the aorta and/or the aortic valve.

According to a further aspect of the present invention there is provided a method of treating heart conditions, disease and/or defects affecting the aorta and/or the aortic valve, comprising the step of implanting the synthetic aortic conduit as described above in the heart of a subject in need of treatment.

The method of treatment is especially intended for use in heart surgery for human patients, but is not necessarily limited thereto.

The method of medical treatment will normally be conducted to alleviate heart conditions, heart disease and/or defects, in particular where the aortic root is to be replaced.

According to a further aspect of the present invention there is provided an aortic root replacement prosthesis comprising an aortic valve and the synthetic aortic conduit described above.

In one embodiment the aortic valve is a tissue graft, for example a xenograft. Advantageously the aortic root replacement prosthesis comprises the synthetic aortic conduit attached to a xenograft aortic valve. Suitably the tissue graft is stabilised against degradation, typically by cross-linking the surface of the tissue graft, suitably by contact with glutaraldehyde and/or formaldehyde.

In a further embodiment the aortic valve is a mechanical graft.

The synthetic aortic conduit may be attached to the aortic valve using any convenient method such as glueing, sewing, clipping (particularly using a mechanical clip-type arrangement) or a combination thereof.

In one embodiment the synthetic aortic conduit is sewn onto the aortic valve.

Optionally, the synthetic aortic conduit is shaped to mimic the sinuses of Valsalva, as described in U.S. Pat. No. 6,852,554 and/or EP 955019.

According to a further aspect of the present invention there is provided an aortic root replacement prosthesis as described above for use in therapy, particularly for use in the treatment of heart conditions, heart disease and/or defects, suitably those which affect the aorta and/or the aortic valve.

According to a further aspect of the present invention there is provided a method of treating heart conditions, disease and/or disorders affecting the aorta and/or the aortic valve, comprising the step of implanting the aortic root replacement prosthesis as described above in the heart of a subject in need of treatment.

The method of treatment is especially intended for use in heart surgery for human patients, but is not necessarily limited thereto.

The method of medical treatment will normally be conducted to alleviate heart conditions, heart disease and/or defects, in particular where the aortic root is to be replaced.

According to a further aspect of the present invention there is provided a package comprising the aortic root replacement prosthesis held in a preservative solution within a leak-resistant container. The package is especially suitable for storage of the aortic root replacement prosthesis until required.

The preservative solution is typically a solution of glutaraldehyde and/or formaldehyde.

The present invention will now be described by way of example only, with reference to the following figures in which.

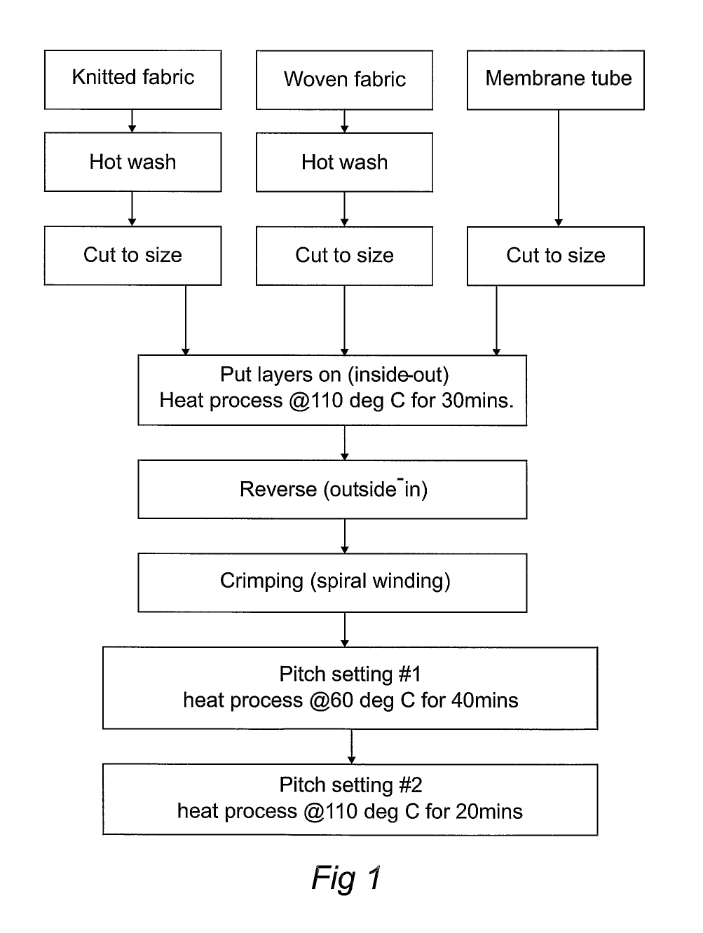

FIG. 1 shows a flow chart of an exemplary method of manufacture of an aortic conduit according to the invention.

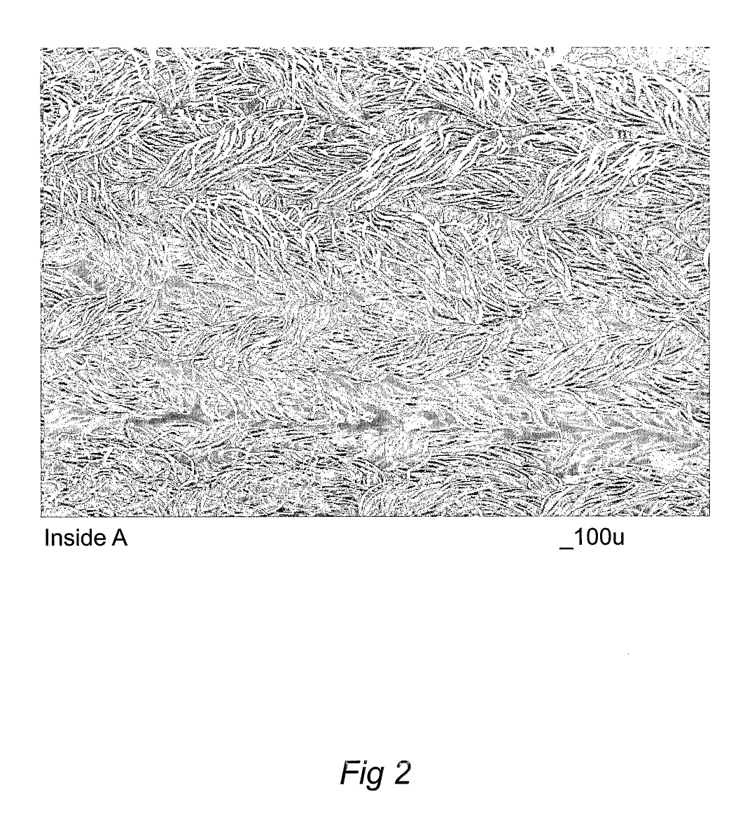

FIG. 2 is an SEM of the first inner tubular layer of an exemplary conduit according to the invention, the first inner tubular layer being formed of knitted polyester.

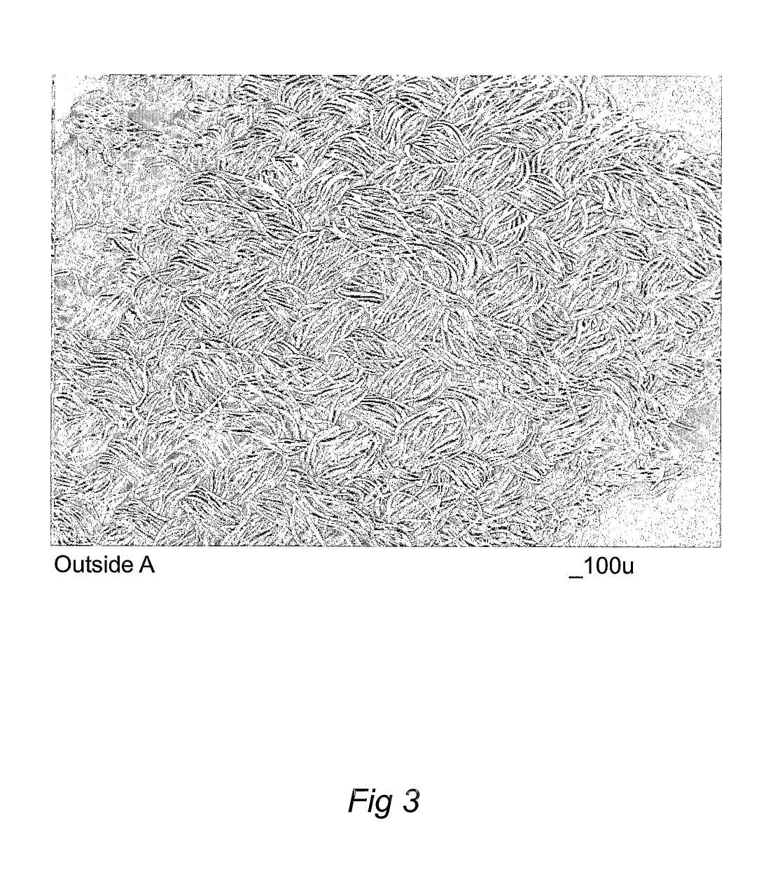

FIG. 3 is an SEM of a second outer tubular layer of an exemplary conduit according to the invention, the second outer tubular layer being formed of woven polyester.

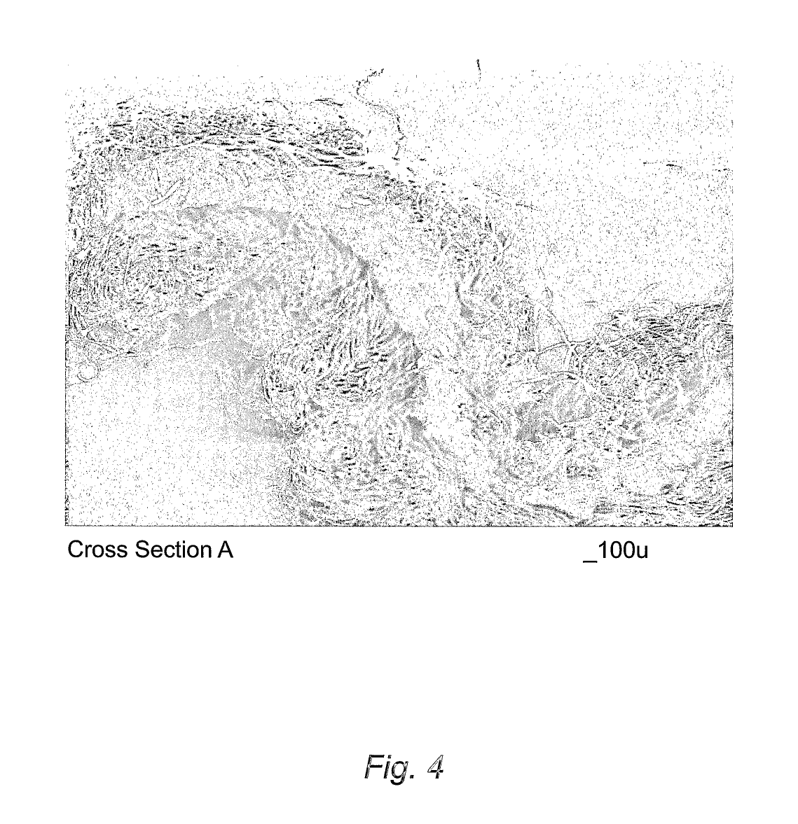

FIG. 4 is an SEM of a cross-section of the conduit shown in FIGS. 2 and 3.

A synthetic aortic conduit is formed comprising a composite structure having an inner luminal layer of woven polyester, an outer tubular layer of knitted polyester and a SEPS non-bioresorbable sealant layer intersposed therebetween. The graft is crimped by the use of a helically wound beading which is removed after the crimp has been heat set, using the step set out below:
1) Hot wash the outer (woven) and the inner (knitted) fabrics.
2) Turn the 19 mm outer fabric (woven) inside-out.
3) Place outer layer on 18 mm production mandrel. Ensure line is straight.
4) Expand 18 mm×0.2 mm SEPS/Squalane membrane and place on graft.
5) Place 18 mm inner layer over membrane, using roll method.
6) Stretch and tape to mandrel. Use cable ties to secure.
7) Place in oven at 110° C. for 30 minutes to melt bond membrane to fabrics.
8) Remove from mandrel and reverse.
9) Crimp on crimping machine.
10) Set crimp in autoclave, Maximum temp 136° C. for 30 mins.
11) Remove from mandrel and cool wash.

The synthetic aortic conduit is sewn to a xenograft aortic valve to form an aortic root replacement prosthesis.

### EXAMPLE 1

An synthetic aortic conduit according to the invention was formed. The synthetic aortic conduit comprises first and second tubular layers formed from knitted polyester, and a bioresorbable membrane interposed between the first and second tubular layers.

The synthetic aortic conduit was implanted into patients.

### EXAMPLE 2

Polyester Twillweave (broken twill/system 2 edge) woven fabric in tubular form and having an internal diameter of 28 mm was hot washed and cut into 600 mm lengths. The fabric wall turned inside out and placed onto 800 mm mandrels of 28.2 mm diameter. The fabric was straightened, stretched longitudinally and taped in place. A tubular SEPs membrane of 26 mm inner diameter and 0.23 mm thickness was placed on top of the fabric using a 31 mm vacuum tube. An ePTFE tubular graft of inner diameter 35 mm was then located on top of the membrane, stretched longitudinally and taped to the mandrel. The whole assembly was covered with a 25 mm silicone tube (located using a 500 mm length×32 mm diameter vacuum tube) and then heated to 110° C. for 30 minutes. Once cool, the silicone tube was removed, the ends of the graft untaped and trimmed.

### EXAMPLE 3

The graft of Example 2 was placed onto a 28 mm mandrel and crimped, using conventional technologies. The graft was stretched by 15% prior to heat setting at 130° C. for 20 minutes. Thus a crimped length of 160 mm is stretched to 185 mm. When cool, the graft is stretched to the finished crimp size of 3 mm pitch, clipped to the mandrel and placed in the oven at 90° C. for 15 minutes. The crimp pitch in the relaxed graft was approximately 3 mm.

1. A synthetic aortic conduit comprising:
i) a first inner tubular layer formed of a porous material;
ii) a second outer tubular layer formed of a porous material; and
a non-bioresorbable sealant layer interposed between said first and second tubular layers.

2. The synthetic aortic conduit as claimed in claim 1, wherein said first inner tubular layer is formed from knitted or woven polyester, or ePTFE.

3. The synthetic aortic conduit as claimed in claim 1, wherein said second outer tubular layer is formed from knitted or woven polyester, or ePTFE.

4. The synthetic aortic conduit as claimed in claim 1, wherein said first inner tubular layer has a different porosity to said second outer tubular layer.

5. The synthetic aortic conduit as claimed in claim 1, wherein said first inner tubular layer has a porosity sufficient to permit tissue ingrowth.

6. The synthetic aortic conduit as claimed in claim 1, wherein the first inner tubular layer has a water permeability of 4,000 to 10,000 ml/min/cm² at 120 mmHg pressure.

7. The synthetic aortic conduit as claimed in claim 1, wherein said second outer tubular layer has a porosity sufficient to permit tissue ingrowth.

8. The synthetic aortic conduit as claimed in claim 1, wherein the second outer tubular layer has a water permeability of 1,000 to 3,000 ml/min/cm² at 120 mmHg pressure.

9. The synthetic aortic conduit as claimed in claim 1, wherein the second outer layer is formed from a woven material and the first inner tubular layer is formed from a knitted material.

10. The synthetic aortic conduit as claimed in claim 9 wherein the second outer tubular layer is formed from woven polyester and the first inner tubular layer is formed from knitted polyester.

11. The synthetic aortic conduit as claimed in claim 1, wherein said non-bioresorbable sealant is an elastomeric polymer.

12. The synthetic aortic conduit as claimed in claim 11 wherein said non-bioresorbable sealant is a silicone, a polyurethane or polyester thermoplastic elastomer, or a styrene-olefin block-polymer.

13. The synthetic aortic conduit as claimed in claim 12 wherein said non-bioresorbable sealant is a styrene-ethylene-propylene-styrene copolymer (SEPS).

14. The synthetic aortic conduit as claimed in claim 1, wherein said synthetic aortic conduit is shaped to mimic the sinuses of Valsalva.

15. The synthetic aortic conduit as claimed in claim 1, wherein said synthetic aortic conduit is crimped.

16. A method of forming a synthetic aortic conduit, said method comprising:
a) interposing a non-bioresorbable sealant layer between a first tubular layer and a second tubular layer to form a composite structure;
b) heating the composite structure of step a) at 50 to 150° C. for 15 to 60 minutes to form the synthetic conduit.

17. The method as claimed in claim 16 wherein in step b) the composite structure is heated to 110° C. for 30 minutes.

18. The method as claimed in claim 16, wherein the composite structure of step a) is formed inside out and wherein said method includes the step of reversing the composite structure once formed.

19. The method as claimed in claim 16, further including a step of crimping the composite structure.

20. The method as claimed in claim 16, wherein the method comprises:
a) interposing a non-bioresorbable sealant layer between a first tubular layer and a second tubular layer to form a composite structure;
b) heating the composite structure of step a) at 50 to 100° C. for 15 to 60 minutes;
c) reversing the composite structure so the inner tubular layer becomes the outer tubular layer and vice versa;
d) wrapping a spiral winding around the exterior of the composite structure at a first pitch;
e) heating the spirally wound composite structure of step d) at 40 to 80° C. for 30 to 60 minutes;
f) heating the wrapped composite structure of step e) at 100 to 150° C. for 10 to 30 minutes; and
g) removing the spiral winding from the composite structure.

21. (canceled)

22. A method of treating at least one of heart conditions, diseases affecting the aorta, disorders affecting the aorta, diseases affecting the aortic valve, and disorders affecting the aortic valve, comprising the step of implanting the synthetic aortic conduit as claimed in claim 1 in the heart of a subject in need of such treatment.

23. The method as claimed in claim 22 wherein said subject is a human.

24. An aortic root replacement prosthesis comprising an aortic valve and a synthetic aortic conduit as claimed in claim 1.

25. The aortic root replacement prosthesis as claimed in claim 24, wherein the aortic valve is a tissue valve.

26. The aortic root replacement prosthesis as claimed in claim 25 wherein the aortic valve is a xenograft valve.

27. The aortic root replacement prosthesis as claimed in claim 25, wherein the tissue valve is stabilised against degradation.

28. The aortic root replacement prosthesis as claimed in claim 27 wherein stabilisation of the tissue valve is achieved by contacting the valve with at least one of glutaraldehyde and formaldehyde.

29. The aortic root replacement prosthesis as claimed in claim 24, wherein said synthetic aortic conduit is attached to the aortic valve by glueing, sewing, clipping or a combination thereof.

30. (canceled)

31. A method of treating at least one of heart conditions, diseases affecting the aorta, disorders affecting the aorta diseases affecting the aortic valve, and disorders affecting the aortic valve, comprising the step of implanting the aortic root replacement prosthesis as claimed in claim 24 in the heart of a subject in need of such treatment.

32. The method as claimed in claim 31 wherein said subject is a human.

33. A package comprising aortic root replacement prosthesis held in a preservative solution within a leak-resistant container.

34. The package as claimed in claim 33 wherein said preservative solution is a solution of at least one of glutaraldehyde and formaldehyde.
